(19) United States
(12) Patent Application Publication
(10) Pub. No.: US 2012/0226325 A1
(43) Pub. Date: Sep. 6, 2012

(54) Fastener Element Retention Feature for Bone Anchors

(75) Inventors: Lauren I. Lyons, San Francisco, CA (US); Michael A. Smith, San Jose, CA (US); Phyllis J. Elson, San Mateo, CA (US)

(73) Assignee: WARSAW ORTHOPEDIC, INC., Warsaw, IN (US)

(21) Appl. No.: 13/040,992

(22) Filed: Mar. 4, 2011

(51) Int. Cl. A61B 17/86

(52) U.S. Cl. 606/305; 606/301

(57) ABSTRACT

A bone anchor includes a distal bone engaging portion, a proximal portion extending from the distal bone engaging portion that includes a thread profile, and a mating element that threadingly engages the thread profile of the proximal portion. A retention feature between the mating element and the proximal portion deformably engages at least one of the mating element and the proximal portion to resist the mating element from moving relative to the proximal portion of the bone anchor.

### BACKGROUND

The present application relates to systems and devices implanted in surgery in a patient, and more particularly but not exclusively relates to systems, devices and techniques for retaining fastener elements of bone anchors to prevent undesired movement of the fastener elements relative to one another post-implantation in the patient.

The human spine serves many functions. The vertebral members of the spinal column protect the spinal cord. The spinal column also supports other portions of the human body. Vertebral implants are often used in the surgical treatment of spinal disorders such as degenerative disc disease, disc herniations, curvature abnormalities, and trauma. Many different types of treatments are used. In some cases, stabilization of one or more vertebral levels of the spinal column involves securing one or more bone anchors to bony structure of the spinal column. The one or more bone anchors can be subjected to various forces or conditions post-implantation that could result in one or more components of the bone anchor to be displaced from their initially implanted configuration. As a result, the effectiveness of the stabilization forces provided by the bone anchor may be reduced or compromised. Thus, there remains a need for further improvements in the devices employed in spinal stabilization techniques.

### SUMMARY

In one embodiment of the present application, a bone anchor includes a distal bone engaging portion, a proximal portion extending from the distal bone engaging portion that includes a thread profile, and a mating element that threadingly engages the thread profile of the proximal portion. A retention feature between the mating element and the proximal portion deforms to engage at least one of the mating element and the proximal portion of the bone anchor to resist the mating element from unthreading from the proximal portion of the bone anchor. In one particular embodiment, deformation of the retention element is caused when the mating element is threadingly engaged to the proximal portion, and the retention element increases the force or torque required to thread and unthread the mating element relative to the proximal portion of the bone anchor.

Another embodiment of the present application comprises a unique bone anchor for performing spinal stabilization in a patient. An additional embodiment of the present application comprises a unique bone anchor with a retention feature that prevents or resists displacement of the mating element relative to the bone anchor. In still another embodiment, a method for promoting spinal stabilization includes deforming a retention element between a mating element that is secured to a bone anchor and a portion of the bone anchor to which the mating element is secured to prevent or resist displacement of the mating element relative to the bone anchor.

Other embodiments include unique methods, systems, devices, kits, assemblies, equipment, and/or apparatus for use in connection with anchors for bony structures. However, in other embodiments, different forms and applications are also envisioned.

Further embodiments, forms, features, aspects, benefits, objects and advantages of the present application will become apparent from the detailed description and figures provided herewith.

### BRIEF DESCRIPTION OF THE FIGURES

FIG. 1 is an exploded assembly view of a bone anchor assembly.

FIG. 2 is a perspective view of a proximal portion of the bone anchor assembly of FIG. 1 mated together.

FIG. 3 is a perspective view of the portion of the bone anchor assembly of FIG. 2 with a mating element of the bone anchor assembly in hidden lines to show the retention element and the proximal portion of the bone anchor.

FIG. 4 is a plan view of the mating element of the bone anchor assembly of FIG. 1.

FIG. 5 is a section view of the mating element through line 5-5 of FIG. 4.

FIG. 6 shows one implantation location and arrangement for the bone anchor assembly of FIG. 1.

FIG. 7 is a perspective view of another embodiment mating element and retention element for a bone anchor assembly.

FIG. 8 is a perspective view of yet another embodiment mating element and retention element for a bone anchor assembly.

FIG. 9 is a perspective view of a further embodiment mating element and retention element for a bone anchor assembly.

FIG. 10 is a perspective view of another embodiment mating element and retention element for a bone anchor assembly.

FIG. 11 is a perspective view of another embodiment mating element and retention element for a bone anchor assembly.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

The present application describes a bone anchor assembly with a retention element to resist or prevent movement of two or more components of the bone anchor assembly relative to one another. In one embodiment, the bone anchor assembly includes a distal bone engaging portion, a proximal portion extending from the distal bone engaging portion that includes a thread profile, and a mating element that threadingly engages the thread profile of the proximal portion. A retention feature deformably engages at least one of the mating element and the proximal portion to resist the mating element from unthreading from the proximal portion of the bone anchor.

In one embodiment, the retention feature includes at least one deformable element that is carried by one of the mating element and the proximal portion of the bone anchor, and protrudes therefrom so it contacts the thread profile of the other of the mating element and proximal portion as the mating element is engaged to the proximal portion. The deformable element increases resistance to threading and unthreading of the mating element and proximal portion relative to one another so that after the mating element is secured in the desired position with a driving instrument, the deformable element resists unthreading of the two components relative to one another post-implantation.

In one specific embodiment, the retention feature is a cylindrical pin that is elongated in directions paralleling a longitudinal axis along which the mating element and proximal portion are threadingly moved relative to one another. In a further variation of this embodiment, the pin is elongated in a direction transverse to the longitudinal axis. The pin can include a cross-section that is circular, square, rectangular, D-shaped, oval, non-circular, polygonal, or irregular. In other embodiments, the pin is not elongated but includes a spherical or cubical shape in shape.

The retention feature can also include one element or more than one element. If multiple elements are employed, the elements are discrete and spaced from one another. The discrete elements can be spaced regularly around longitudinal axis, irregularly around the longitudinal axis, along the longitudinal axis, and combinations thereof. In a further embodiment, the retention feature extends substantially around the entire thread profile of the mating element and proximal portion when engaged to one another. The retention feature can be secured to the bone anchor assembly by a press fit, an adhesive, a snap fit, a fastener, or allowed to simply reside in the receptacle without a substantial securing arrangement with the portion of the bone anchor assembly in which it resides.

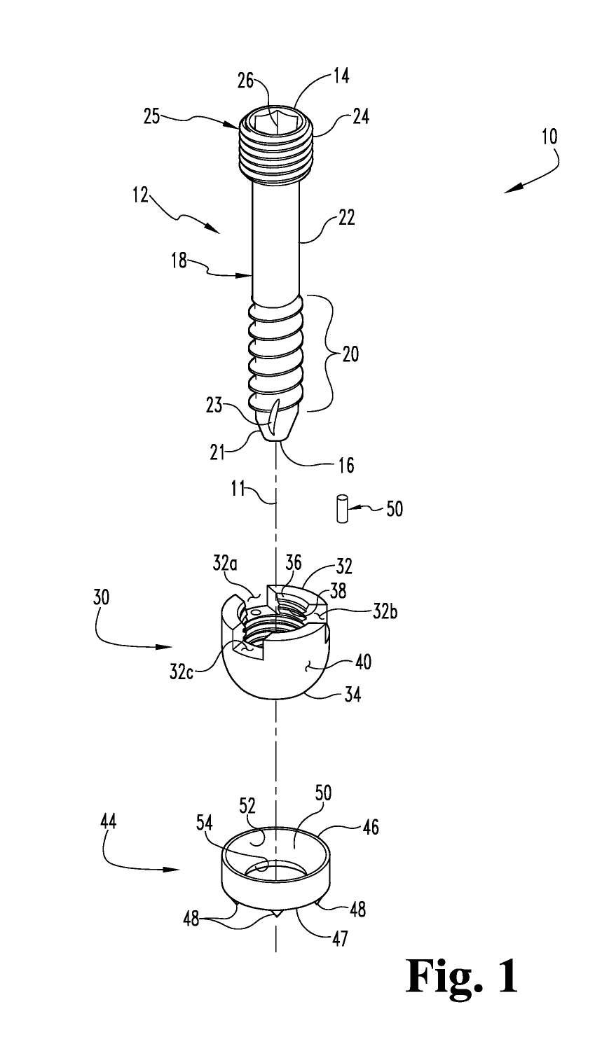

Referring generally to FIG. 1, a bone anchor assembly 10 extends along a longitudinal axis 11 and includes a distal bone anchor 12, a mating element 30, and an engaging member 44. As will be discussed in greater detail below, the mating element 30 and the engaging member 44 are positionable along the bone anchor 12. The bone anchor 12 extends along longitudinal axis 11 between a proximal end 14 and a distal end 16 and includes an elongated shaft 18 extending distally from a proximal mounting portion 25. The elongated shaft 18 includes a distal threaded portion 20 that is configured to engage with bone or bony tissue. More particularly, in the illustrated form, the distal threaded portion 20 includes a tapered or pointed end section 21 to facilitate entry into bone. However, in other embodiments, the distal threaded portion 20 may define a blunt or rounded distal end. The distal threaded portion 20 may also be provided with a cutting flute 23 extending proximally from the distal end 16 to provide the bone anchor 12 with self-cutting or self-tapping capabilities to facilitate its advancement into bone.

In still other embodiments, the bone anchor 12 is threaded along all or a substantial portion of its length. Bone anchor 12 may also include a distal bone engaging portion that is non-threaded, spiked, or hook-shaped, for example, or include any suitable configuration to engage bony structure. In addition, the bone anchor 12 may include a solid shaft 18, or a shaft 18 that includes a passageway that extends between and opens at the proximal end 14 and the distal end 16. The passageway can generally be sized and configured to allow placement of the bone anchor over a guidewire. Additionally or alternatively, the passageway may communicate with fenestration openings (not shown) that may be used to deliver material such as, for example, bone cement from the passageway and into areas of bone adjacent the bone anchor 12.

A non-threaded portion 22 extends between the distal threaded portion 20 and the proximal mounting portion 25, although embodiments where the distal threaded portion 20 extends to the proximal mounting portion 25 are also contemplated. The proximal mounting portion 25 includes external threading 24 extending radially outwardly from non-threaded portion 22 and an internal driving print 26 which may be non-circular such as, for example, hexagonal or rectangular shaped, to provide non-rotational engagement between the proximal mounting portion 25 and a driving instrument (not shown) to engage the bone anchor 12 and rotate it into bone or bony tissue. Examples of non-circular configurations for the driving print 26 include but are not limited to slotted, Phillips, hexagonal, Torx, spline drive, and double hex configurations.

Proximal mounting portion 25 is illustrated in FIG. 1 as a circular cylindrical member with an external thread profile 24 around longitudinal axis 11. Mounting portion 25 is fixed in position relative to shaft 18. In other embodiments, mounting portion 25 is rotatable and/or pivotal relative to shaft 18. In still other embodiments, mounting portion 25 includes a U-shaped, saddle-like configuration to receive a spinal rod or other spinal stabilization element. In any embodiment, mounting portion 25 can be externally threaded as shown, include an internal thread profile, or include a combination of an internal and external thread profiles.

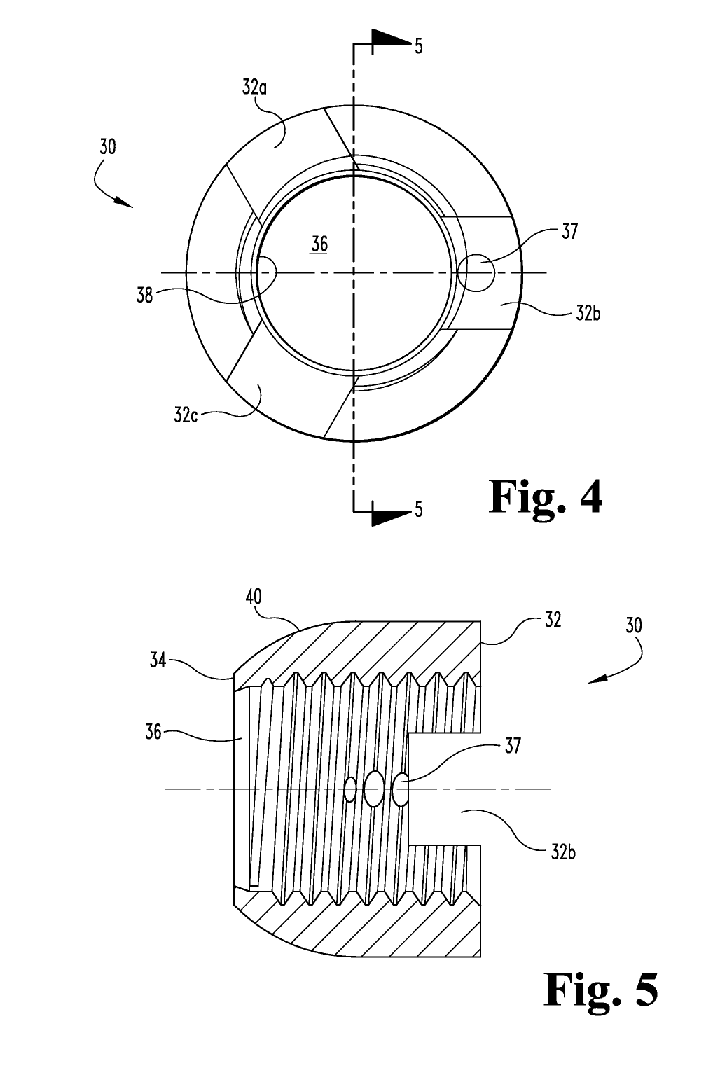

The mating element 30, as also shown in FIGS. 4-5, extends between a proximal end 32 and a distal end 34 and includes a passage 36 that extends between and opens at the proximal end 32 and the distal end 34. The passage 36 includes internal threading 38 around longitudinal axis 11 configured to cooperate and engage with the external thread profile 24 of the mounting portion 25. Similarly, when the mating element 30 is engaged with the external thread profile 24 and rotated relative to the bone anchor 12, its relative axial position along the length of the bone anchor 12 is changed. The mating element 30 also includes an arcuately convexly rounded external portion 40 extending proximally from the distal end 34 such that the mating element 30 includes a partially spherical external configuration. Additionally, the proximal end 32 also includes a plurality of notches 32a, 32b and 32c that are configured to engage the mating element 30 by a driver instrument (not shown) suitably configured for rotating the mating element 30 about the bone anchor 12. Mating element 30 further includes a receptacle 37 extending therein to receive retention element 50, as discussed further below. Receptacle 37 opens proximally at the proximal face of the recessed surface in one of notches 32a, 32b, 32c, such as notch 32b in the illustrated embodiment, and extends to a blind end in mating element 30. Receptacle 37 parallels longitudinal axis 11, and opens through thread profile 38 to directly communicate with passage 36.

The engaging member 44 extends between a proximal end 46 and a distal end 47, with a plurality of engaging members in the form of spikes 48 extending from the distal end 47. In other forms, the distal end 47 can be provided with teeth, knurling, grooves or other types of engaging features in addition to or in lieu of the spikes 48. The engaging member 44 also includes a passage 50 that extends between and opens at the proximal end 46 and the distal end 47. The passage 50 includes an arcuately rounded internal portion 52 extending distally from the proximal end 46 such that the engaging member 44 includes a partially spherical internal configuration. The passage 50 also defines an inner opening dimension at distal portion 54 that is generally greater than the outer dimension of the non-threaded portion 22 of the bone anchor 12 such that the engaging member 44 can be moved along the bone anchor 12 and pivoted relative to the bone anchor 12.

When the bone anchor assembly 10 is assembled, the engaging member 44 may be positioned along the shaft 18 of the bone anchor 12 distally of the mounting portion 25, and the arcuately rounded portion 40 of the mating element 30 may be positioned in the arcuately rounded portion 52 of the engaging member 44. In this arrangement, the interaction between the arcuately rounded portions 40, 52 and the ability for the engaging member 44 to pivot relative to the bone anchor 12 facilitates multi-axial positioning of the engaging member 44 in a plurality of planes that extend transversely to the plane of the mating element 30 when engaged with the external thread profile 24 of the mounting portion 25. Similarly, this adjustability facilitates use of the bone anchor assembly 10 in connection with bones that are oriented at an angle relative to the mounting portion 25 of bone anchor 12. Moreover, while not previously discussed, it should be appreciated that bone anchor assembly 10 can be implanted across adjacent bones or bone pieces and used to draw the adjacent bones or bone pieces toward one another. More particularly, once the distal threaded portion 20 of the bone anchor 12 is engaged with a first one of the bones or bone pieces, the mating element 30 can be rotated and distally advanced relative to the bone anchor 12 in order to bring the engaging member 44 into contact with a second one of the bones or bony pieces. As the mating element 30 is further rotated in this manner, the bones or bony pieces are drawn together and any gap positioned therebetween may be reduced or eliminated. However, it should be understood that the bone anchors described herein have application in any suitable spinal stabilization or other bone anchoring procedure.

While not previously discussed, it should be appreciated that the bone screw 12, the mating element 30, and the engaging member 44 may be formed from any suitable biocompatible material, including but not limited to titanium, titanium alloy, stainless steel, metallic alloys, polyaryletherketone (PAEK), polyetheretherketone (PEEK), carbon-reinforced PEEK, polyetherketoneketone (PEKK), polysulfone, polyetherimide, polyimide, ultra-high molecular weight polyethylene (UHMWPE), and plastics, just to name a few possibilities. It is further contemplated that retention element 50 is made from a material that deforms more readily than the material comprising at least the threads of mating element 30 and mounting portion 25. In particular, in the illustrated embodiment retention element 50 deforms as a result of contact with thread profile 24 of mounting portion 25 when mating element 30 is advanced sufficiently along mounting portion 25 to locate retention element 50 along thread profile 24.

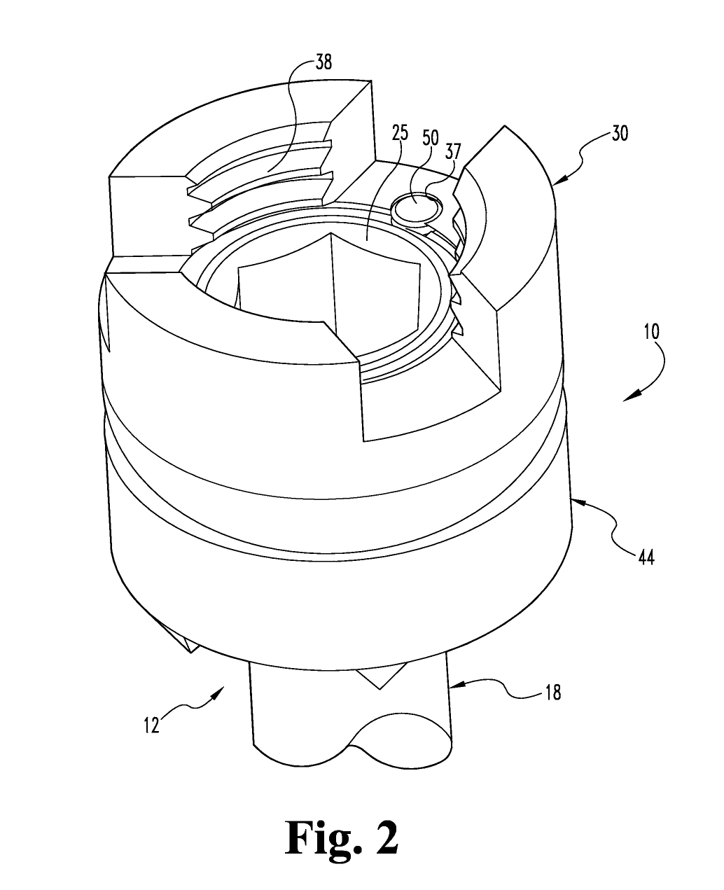

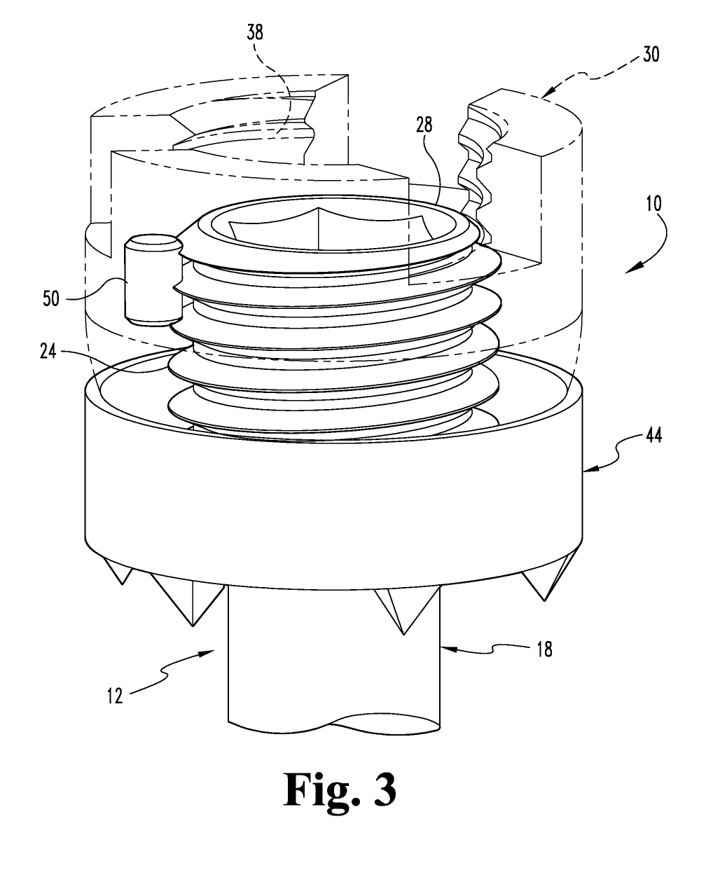

Referring now to FIGS. 2-3, when mating element 30 is secured to mounting portion 25, retention element 50 is housed in receptacle 37 and directly contacts mounting portion 25 to resist or prevent movement of mating element 30 relative to mounting portion 25. Since retention element 50 projects into passage 36 of mating element 30, thread profile 24 deforms retention element 50 and embeds into retention element 50 to provide frictional engagement with thread profile 24 of bone anchor 12. Retention element 50 provides interference with the thread profile 24 to prevent or resist reverse rotation of mating element 30 after mating element 30 is tightened onto bone anchor 12 in situ. In the illustrated embodiment, retention element 50 includes a smooth pin-shaped body that is elongated in the direction along longitudinal axis 11 so that it directly contacts multiple thread crests along thread profile 24. In an alternative embodiment, retention element 50 can be formed with an imprint of thread profile 38 to receive thread profile 24 as mating element 30 is threadingly advanced along mating portion 25. This pre-threaded arrangement of retention element 50 could assist in preventing retention element 50 from rotating or spinning in receptacle 37 as mating element 30 is threadingly advanced along mating portion 25.

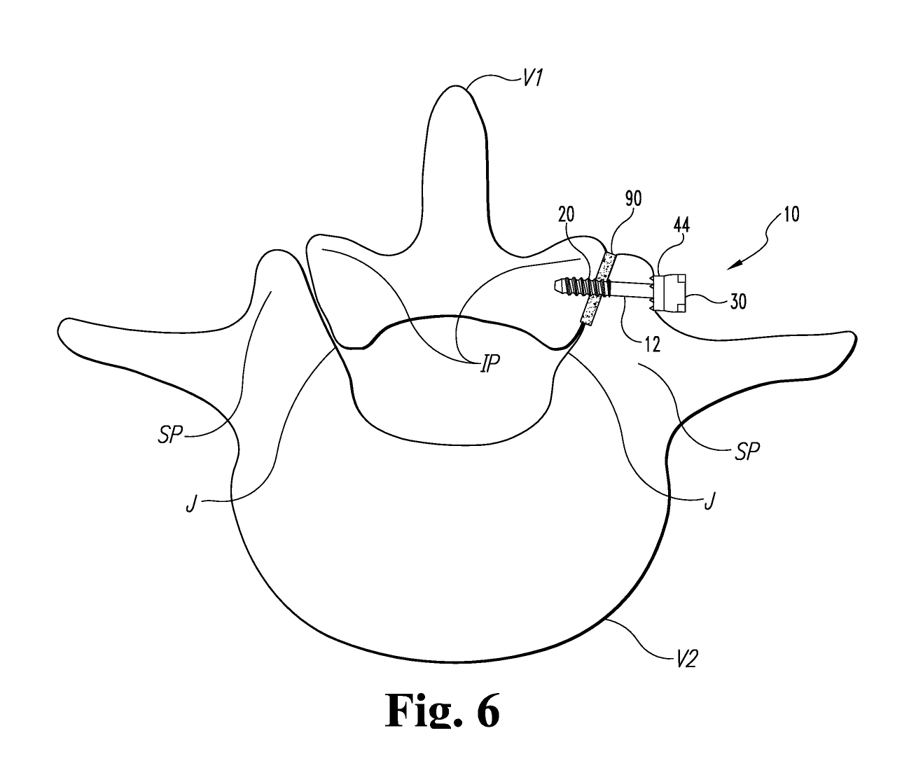

Referring now to FIG. 6, one potential implantation location method for bone anchor assembly 10 is shown. It should be understood, however, that bone anchor assembly has application in other locations along the spinal column and with other bone structures. For example, bone anchor assembly 10 could be engaged to the pedicle of a vertebra and configured to receive a spinal rod extending along the spinal column. In FIG. 6, bone anchor assembly 10 is positioned across the facet joint J where an implant 90 has been implanted. Bone anchor assembly 10 can be positioned across the facet joint J. More particularly, the bone anchor 12 can be engaged so that it extends through the superior articular process SP of the vertebra V2 and into/through the implant 90. The distal threaded portion 20 of the bone anchor 12 can then be engaged with the inferior articular process IP of the vertebra V1 adjacent to vertebra V2 to secure the bone anchor 12 in bone. Once the distal threaded portion 20 engages the inferior articular process IP of the vertebra V1, the mating element 30 can be distally advanced relative to the bone anchor 12 to contact and force the engaging member 44 against the exterior surface of the superior articular process SP of the vertebra V2. As the mating element 30 is advanced in this manner, the superior and inferior articular processes SP, IP are drawn together and a clamping force is exerted onto the implant 90 positioned therebetween. This clamping force, in combination with the bone anchor 12 extending through the implant 90, retains the implant 90 within the facet joint J. However, in other non-illustrated forms where the bone anchor 12 does not extend through the implant 90, the clamping force alone exerted by the superior and inferior articular processes SP, IP retains the implant 90 within the facet joint J. In any event, retention element 50 prevents or resists mating element 30 from unthreading or otherwise backing off of bone anchor 12 due to forces or bone conditions occurring at the implantation location after the surgery is complete.

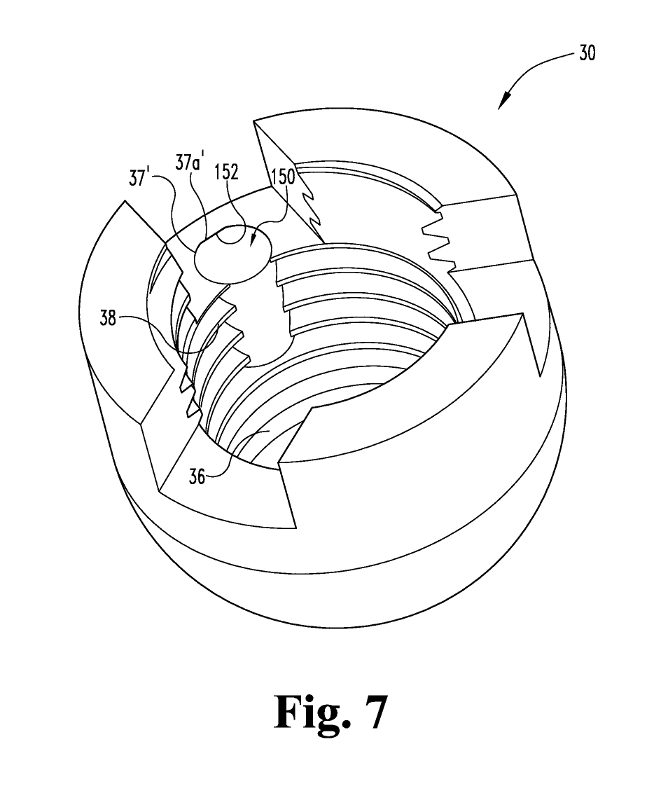

Referring to FIG. 7, there is shown mating element 30 with another embodiment retention element designated at 150. Retention element 150 includes an elongated body extending along longitudinal axis 11 in a modified receptacle 37′. Retention element 150 includes a flat side 152 that interfaces with a flat side 37a′ of receptacle 37′ to prevent or resist rotation of retention element 150 in receptacle 37′ as mating element 30 is threadingly advanced along mating portion 25. The opposite side of retention element 50 overlaps with thread profile 38 to extend into passage 26 and engage thread profile 24 of mating portion 25 of bone anchor 12. In the illustrated embodiment, retention element 150′ includes a D-shaped cross-section normal to its length. In other embodiments, retention element 150 includes any suitable non-circular shape to prevent it from rotating in receptacle 37′.

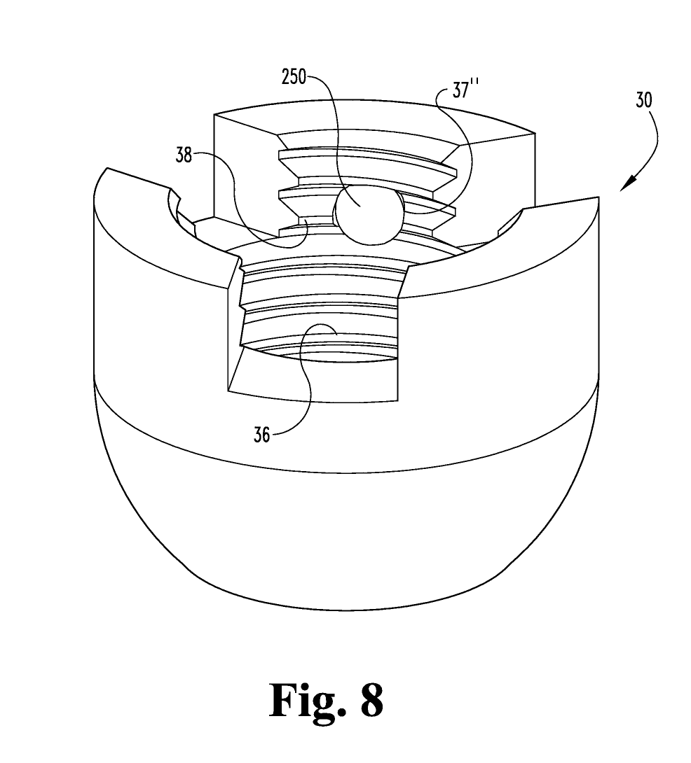

Referring to FIG. 8, there is shown mating element 30 with another embodiment retention element designated at 250. Retention element 250 can include an elongated cylindrical body like retention element 50 discussed above. However, retention element 250 is press-fit or otherwise positioned in another modified receptacle 37″ that extends generally normal to longitudinal axis 11. Receptacle 37″ includes a blind end (not shown) in mating element 30 and an opposite end that opens into passage 36 so that retention element 250 overlaps with thread profile 38 to engage with the thread profile 24 of mounting portion 25 of bone anchor 12. Other embodiments contemplate other shapes for retention element 250, including spherical, cubic, and non-cylindrical shapes.

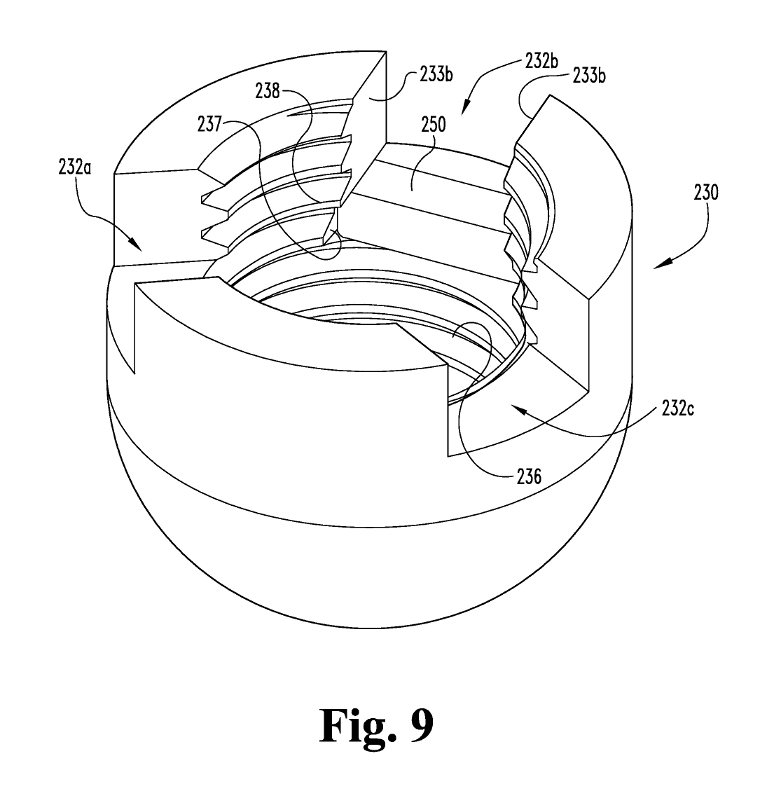

In still other embodiments, it is contemplated that other arrangements are provided for securing the retention element to the mating element. For example, in FIG. 9 there is shown another embodiment mating element 230 that is identical to mating element 30 discussed above but is without a receptacle 37. Mating element 230 also includes a slot 237 that is configured to receive another embodiment retention element 250 that is press fit, adhered or otherwise secured in at least one of notches 232a, 232b, 232c, such as notch 232b in the illustrated embodiment. Mating element 230 is positioned in overlapping arrangement with thread profile 238 so that it projects into passage 236 to engage thread profile 24 of mounting portion 25 of bone anchor 12. Slot 237 is arranged to undercut sidewalls 233b of notch 232b. Retention element 250 includes a block or bar-shaped body with a rectangular cross-section normal to its length. Retention element 250 is oriented lengthwise to extend between sidewalls 233b and its opposite ends are positioned in slot 237 to frictionally engage retention element 250 to mating element 230. Additionally or alternatively, retention element 250 can be secured to mating element 230 with adhesive or fasteners.

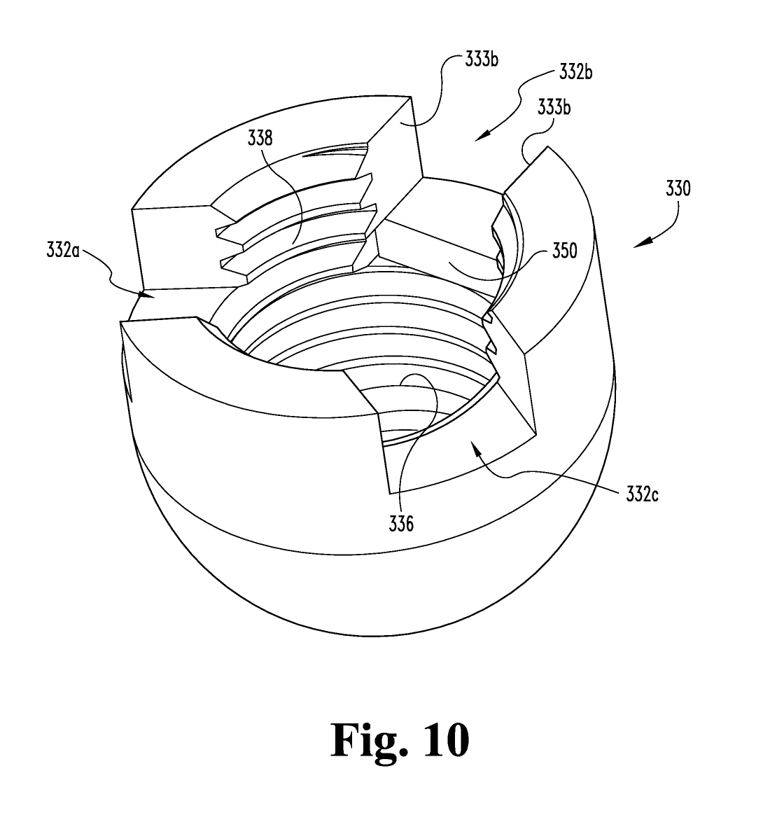

In FIG. 10 there is shown another embodiment mating element 330 that is identical to mating element 30 discussed above but is without a receptacle 37. Mating element 330 receives retention element 350 that provides a shim that is press fit, adhered or otherwise secured between sidewalls 333b of notch 332b. Of course, it is to be understood that shim 350 or one or more other shims can be placed in the other notches 332a, 332c. Retention element 350 extends in overlapping arrangement with thread profile 338 so that it projects into passage 336 to engage thread profile 24 of mounting portion 25 of bone anchor 12. Retention element 350 includes a block or bar-shaped body with a rectangular cross-section normal to its length. Retention element 350 is oriented lengthwise to extend between sidewalls 333b and its opposite ends are positioned against sidewalls 333b to frictionally engage retention element 350 to mating element 330. Additionally or alternatively, retention element 350 can be secured to mating element 330 with adhesive or fasteners.

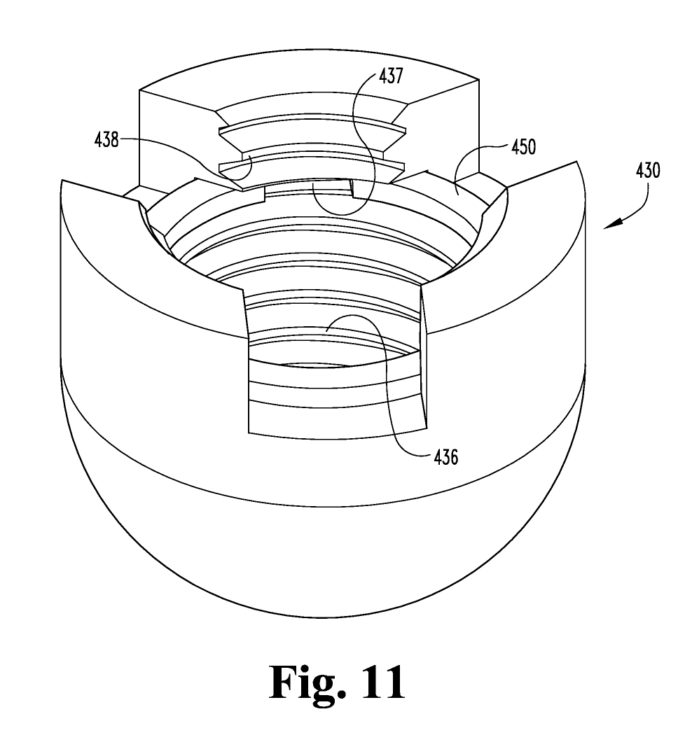

In another example, in FIG. 11 there is shown another embodiment mating element 430 that is identical to mating element 30 discussed above but is without a receptacle 37. Mating element 430 also includes a groove 437 that is configured to receive an embodiment of retention element 450 that is a snap ring with a notch in one side that allows it to be radially compressed for insertion into passage 436 and released to expand radially outwardly in groove 437. Retention element 450 extends into thread profile 438 and overlaps passage 436 to engage the thread profile along mating portion 25 when mating element 430 is engaged thereto.

Additionally, the instruments, devices, systems, techniques and methods described herein may also be used in surgical procedures involving animals, or in demonstrations for training, education, marketing, sales and/or advertising purposes. Furthermore, the instruments, devices, systems, techniques and methods described herein may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words/phrases such as “a”, “an”, “at least one”, and/or “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. A bone anchor assembly, comprising:
a bone anchor including a distal bone engaging portion and a proximal mounting portion defining a thread profile extending around a longitudinal axis;
a mating element threadingly engageable to said thread profile of said mounting portion; and
a retention element positionable between said mating element and said proximal mounting portion in deformable engagement with said thread profile of said mounting portion to resist said mating element from unthreading from said mounting portion.

2. The bone anchor assembly of claim 1, wherein said mating element includes a receptacle extending along said longitudinal axis and said retention element is positioned in said receptacle along said longitudinal axis.

3. The bone anchor assembly of claim 2, wherein said mating element includes an inner thread profile extending around a passage that extends through a proximal end and an opposite distal end of said mating element, said receptacle being in direct communication with said passage through said inner thread profile so that when said mating element is threadingly engaged to said mounting portion said retention element contacts said thread profile of said mounting portion.

4. The bone anchor assembly of claim 3, wherein said mating element includes a sidewall defining at least one notch that extends proximally into said sidewall, and said receptacle opens at a proximally facing surface of said notch.

5. The bone anchor assembly of claim 4, wherein said retention element includes an elongated body that protrudes from said receptacle and into said inner thread profile of said mating element.

6. The bone anchor assembly of claim 1, wherein said retention element is made from a material that deforms around said thread profile of said mounting portion as said mating element is threadingly advanced along said mounting portion of said bone anchor.

7. The bone anchor assembly of claim 1, wherein said mating element includes a receptacle extending generally normal to said longitudinal axis and said retention element is positioned in said receptacle in a generally normal orientation to said longitudinal axis.

8. The bone anchor assembly of claim 1, wherein:
said mating element includes an inner thread profile that extends around a passage that extends through a proximal end and an opposite distal end of said mating element, said mating element further including a sidewall defining at least one notch that extends proximally into said sidewall from said proximal end of said mating element, said notch including a proximally facing surface and opposite sidewalls that extend from said proximally facing surface to said proximal end of said mating element, said mating element further including a slot extending under said sidewalls; and
said retention element includes an elongate body positioned in said slot and projecting into said passage of said mating element so that said retention element engages said thread profile of said mounting portion when said mating element is threadingly engaged to said mounting portion of said bone anchor.

9. The bone anchor assembly of claim 1, wherein:
said mating element includes an inner thread profile that extends around a passage that extends through a proximal end and an opposite distal end of said mating element, said mating element further including a sidewall defining at least one notch that extends proximally into said sidewall from said proximal end of said mating element, said notch including a proximally facing surface and opposite sidewalls that extend from said proximally facing surface to said proximal end of said mating element; and
said retention element includes an elongate body positioned in said notch with opposite ends of said retention element in engagement with said sidewalls and projecting into said passage of said mating element so that said retention element engages said thread profile of said mounting portion when said mating element is threadingly engaged to said mounting portion of said bone anchor.

10. The bone anchor assembly of claim 1, wherein:
said mating element includes an inner thread profile that extends around a passage that extends through a proximal end and an opposite distal end of said mating element, said mating element further including a groove extending around said passage and into said inner thread profile; and
said retention element includes a body formed as a snap ring positioned in said groove and projecting into said passage of said mating element so that said retention element engages said thread profile of said mounting portion when said mating element is threadingly engaged to said mounting portion of said bone anchor.

11. The bone anchor assembly of claim 1, wherein at least said thread profile of said mounting portion is comprised of a metal material and said retention element is comprised of a polymer material.

12. A bone anchor assembly, comprising:
a bone anchor including a distal bone engaging portion and a proximal mounting portion defining a thread profile extending around a longitudinal axis;
a mating element threadingly engageable to said thread profile of said mounting portion; and
a retention element supported by said mating element and protruding from said mating element so that when said mating element is threadingly engaged to said proximal mounting portion said retention element deformably engages said thread profile of said mounting portion to resist said mating element from unthreading from said mounting portion.

13. The bone anchor assembly of claim 12, wherein said thread profile of said mounting portion is an external thread profile and said mating element includes an internal thread profile extending around a passage that receives said mounting portion when said mating element is threadingly engaged to said mounting portion.

14. The bone anchor assembly of claim 13, wherein said mating element includes a receptacle in which said retention element is positioned, said receptacle opening through said inner thread profile into direct communication with said passage of said mating element.

15. The bone anchor assembly of claim 14, wherein said receptacle is elongated along said longitudinal axis and said retention element includes an elongated body extending along said receptacle to deformably engage multiple crests of said thread profile of said mounting portion when said mating element is threadingly engaged to said mounting portion.

16. The bone anchor assembly of claim 13, wherein said mating element includes a groove extending therein around said inner thread profile and said retention element is a snap ring positioned in said groove.

17. The bone anchor assembly of claim 13, wherein said mating element includes at least one notch extending from a proximal end of said mating element to a proximally facing surface of said notch, said notch including opposite sidewalls extending from said proximally facing surface to said proximal end of said mating element, and said mating element further comprising a slot that extends beneath said sidewalls adjacent to said inner thread profile, and said retention element is positioned in said slot and extends between said sidewalls.

18. The bone anchor assembly of claim 13, wherein said mating element includes at least one notch extending from a proximal end of said mating element to a proximally facing surface of said notch, said notch including opposite sidewalls extending from said proximally facing surface to said proximal end of said mating element, and said retention element is positioned in said notch and is press fit between said sidewalls.

19. The bone anchor assembly of claim 12, wherein said bone engaging portion of said bone anchor includes a threaded shaft extending on said longitudinal axis and said retention element is offset from said longitudinal axis.

20. The bone anchor assembly of claim 19, wherein said bone engaging portion is fixed relative to said proximal mounting portion.
